(19) United States
(12) Patent Application Publication
(10) Pub. No.: US 2019/0287671 A1
(43) Pub. Date: Sep. 19, 2019

(54) SYSTEM, METHOD AND APPARATUS FOR MANAGING SURGICAL PROCEDURES

(71) Applicant: SURGIOLOGY INC., Toronto (CA)

(72) Inventors: Kevin MAKO, Toronto (CA); Son CHUNG, Toronto (CA); Paul DAVIS, Toronto (CA); Avi ORNER, Toronto (CA); Oleg SAFIR, Toronto (CA); Steve CRICHTON, Waterdown (CA)

(73) Assignee: SURGIOLOGY INC., Toronto, ON (CA)

(21) Appl. No.: 16/344,548

(22) PCT Filed: Oct. 24, 2017
(86) PCT No.: PCT/CA2017/051266

§ 371 Date: Apr. 24, 2019

Related U.S. Application Data
(60) Provisional application No. 62/411,953, filed on Oct. 24, 2016.

(51) Int. Cl. G16H 20/40; G16H 10/60

(52) U.S. Cl. CPC G16H 20/40; G16H 10/60

(57) ABSTRACT

The present invention provides a system and method for managing surgical procedures, more particularly, systems and methods for ensuring the correct location/side of a surgery is described. The system includes a medical tag that has multiple elements, including a decal. In a preferred embodiment, the medical tag system of the present invention has one or more components; a first component is to be located or directly associated with the patient, while the second component is to be used with the where the procedure or activity is to take place (e.g. hospital, clinic, etc.). There is also provided a software component for users to access appropriate to medical information.

### INCORPORATION BY REFERENCE

This application claims priority from U.S. provisional application No. 62/411,953 filed Oct. 24, 2016 which is herein incorporated by reference.

### FIELD OF INVENTION

The present invention provides systems, methods and apparatus for managing medical procedures, more particularly, systems and methods for ensuring the correct location/side of a surgery is described.

### BACKGROUND TO THE INVENTION

During surgical procedures, it may be possible for medical personnel to make mistakes because such personnel may incorrectly identify one or more of the following: (1) the patient; (2) the procedure to be performed, and/or (3) site of the procedure to be performed. Specifically, surgeries where the wrong appendage, organ or side is operated on occurs despite existing structural and/or behavioral requirements by medical staff or personnel. Such a mistake may occur because there may be an insufficient connection between (1) the patient (who may be unconscious), (2) the patient's information, and (3) the information about the patient's procedure.

Dramatic consequences can occur when a surgical procedure is performed on the wrong site or surgery location (e.g., left side instead of right side), on the wrong patient, or involves the wrong procedure. Such systems are problematic. However, as they require additional equipment that, for example, may not be compatible with existing hospital equipment or may be prone to improper use by staff.

To reduce the possibility of such errors, medical staff (or the patient) may mark surgical locations. Despite this and other more advanced methods, the above noted medical mistakes persist. Accordingly, a need exists for simple yet effective systems and methods to reduce such errors.

### SUMMARY OF THE INVENTION

The methods, systems and apparatus of the present invention include apparatus for marking a surgery site (e.g., location of surgical incision) or a site of other medical action with a patient and/or procedure specific medical marking (e.g., bar code, QR codes, RFID/NFC tag, magnetic stripe, etc.), which may be used in conjunction with a medical workflow system for managing medical procedures and/or actions.

An embodiment of the present invention is directed to a patient medical information system for use in a medical workflow for conducting a medical procedure on a patient at a location on the patient's body, the patient medical information system comprising: (a) a first medical information component physically associated with the location of the patient's body and providing readable personal health information of the patient; (b) a second medical information component associated with the patient; and (c) a third medical information component associated with a non-patient location and providing readable personal health information of the patient and medical workflow related information.

Another embodiment of the present invention is directed to the above wherein the first medical information component further comprises a strap for wrapping around an extremity of the patient.

Another embodiment of the present invention is directed to the above wherein the first medical information component further comprises markings that provide access to at least one piece of medical information of the patient.

Another embodiment of the present invention is directed to the above wherein the first medical information component further comprises a patient surgical decal and a patient informational decal.

Yet another embodiment of the present invention is directed to the above wherein the surgical decal is applied to the location of the portion of the body of the patient.

Yet another embodiment of the present invention is directed to the above wherein the patient information decal is associated with the second medical information component.

Yet another embodiment of the present invention is directed to the above wherein the markings are selected from the group consisting barcodes, QR codes, RFID/NFC tags, and magnetic stripes.

Yet another embodiment of the present invention is directed to the above wherein the second medical information component is a card providing the patient access to the third medical information component.

Yet another embodiment of the present invention is directed to the above wherein the third medical information component.

Yet another embodiment of the present invention is directed to the above wherein the third medical information component is a web based application.

Another embodiment of the present invention is directed to a medical workflow for conducting a medical procedure on a patient, the medical workflow comprising: (a) a medical professional and the patient confirming that the medical information of the patient associated with a first medical record component, the first medical record component physically associated with the patient, is correct and accurate; (b) confirming the site of the medical procedure based on the location of the first medical record component on the body of the patient; (c) marking the site of the medical procedure based on the location of the first medical record component; and (d) removing the patient medical tag physically from the site of the medical procedure and transferring the information contained therein to a second medical record component.

### BRIEF DESCRIPTION OF THE DRAWINGS

In the drawings, which illustrate embodiments of the invention:

FIG. 1 illustrates an embodiment of the present invention.

FIG. 1A to 1E illustrate further embodiments of the present invention.

FIGS. 2 & 2A illustrates a further embodiment of the present invention.

FIGS. 3 & 3A illustrates a further embodiment of the present invention.

FIGS. 4 & 4A illustrates a further embodiment of the present invention.

FIGS. 5 to 17 illustrate further embodiments of the present invention.

### DESCRIPTION OF THE PREFERRED EMBODIMENTS

The description that follows, and the embodiments described therein, is provided by way of illustration of an example, or examples, of particular embodiments of the principles and aspects of the present invention. These examples are provided for the purposes of explanation, and not of limitation, of those principles and of the invention.

It should be noted that the figures are not drawn to scale and that elements of similar structures or functions may be represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated.

In the following specification, the terms “personal health information”, “patient health information” “protected health information” or “PHI” will be used interchangeably and will be understood by a person skilled in the art to mean health information about or relating to a patient, including but not limited to, information relating to one or more of the following: (a) the physical or mental health of the individual, including information that consists of the health history of the individual's family; (b) the provision of health care to the individual, including the identification of a person as a provider of health care to the individual; (c) relates to payments or eligibility for health care, or eligibility for coverage for health care, in respect of the individual; and (d) a patient identification number.

In the following specification, the terms “health record”, “electronic health record” “HR” or “EHR” will be understood by a person skilled in the art to refer to a collection of PHI in traditional paper form, as well as electronic or digital format that is capable of being shared across different health care information systems, by way of network-connected enterprise-wide information system and other information networks or exchanges. HRs/EHRs provide a range of data, including, but not limited to demographics, medical history, medication and allergies, immunization status, laboratory test results, radiology images, vital signs, personal stats like age and weight, and billing information.

Some portion of the detailed descriptions that follow are presented in terms of procedures, steps, logic block, processing, and other symbolic representations of operations on data bits that can be performed on computer memory. These descriptions and representations are the means used by those skilled in the data processing arts to most effectively convey the substance of their work to others skilled in the art. A procedure, computer executed step, logic block, process, etc. may be here, and generally, conceived to be a self-consistent sequence of operations or instructions leading to a desired result. The operations are those requiring physical manipulations of physical quantities. Usually, though not necessarily, these quantities take the form of electrical or magnetic signals capable of being stored, transferred, combined, compared, and otherwise manipulated in a computer system. It has proven convenient at times, principally for reasons of common usage, to refer to these signals as bits, values, elements, symbols, characters, terms, numbers or the like.

It should be borne in mind, however, that all of these and similar terms are to be associated with the appropriate physical quantities and are merely convenient labels applied to these quantities. Unless specifically stated otherwise as apparent from the following discussions, it may be appreciated that throughout the present invention, discussions utilizing terms such as “receiving,” “creating,” “providing,” or the like refer to the actions and processes of a computer system, or similar electronic computing device, including an embedded system, that manipulates and transfers data represented as physical (electronic) quantities within the computer system's registers and memories into other data similarly represented as physical quantities within the computer system memories or registers or other such information storage, transmission or display devices.

In the following specification, the terms “electronic medical records” or “EMR” will be understood by a person skilled in the art to mean an electronic or digital medical record created or recorded in an organization (e.g. hospital, clinic, insurance provider, etc.) that contains EHRs.

It should also be appreciated that the present invention can be implemented in numerous ways, including as a process, method, an apparatus, a system, a device, a method, or a computer readable medium such as a computer readable storage medium or a computer network wherein program instructions are sent over a network (e.g. optical or electronic communication links). In this specification, these implementations, or any other form that the invention may take, may be referred to as processes. In general, the order of the steps of the disclosed processes may be altered within the scope of the invention.

The systems and methods described herein generally relate to the field of health care, and may be used to warn and prevent health care or medical staff or personnel (e.g. doctors, nurses, nurse practitioners, hospital administration staff, etc.) from inadvertently performing or otherwise taking incorrect action. Such incorrect action may include, a medical action or procedure on the wrong site (etc., limb, portion of a body, etc.), the wrong patient, or the wrong procedure. The system and method described herein may be employed anywhere medical actions or procedures may be conducted or performed, including but not limited to hospitals, acute care centers, emergency rooms, doctor's offices, nursing homes, convalescent hospitals, field hospitals, and all other medical and health-care facilities.

A person skilled in the relevant art will understand that the term “workflow” or “medical workflow”, in the present description, is the series of activities or steps that may be necessary to complete a task. Each step in a workflow has a specific step before it and a specific step after it, with the exception of the first step. In a linear workflow, the first step is usually initiated by an outside event. If the workflow has a loop structure, however, the first step is initiated by the completion of the last step. Medical procedures in general and surgical procedures in particular, involve a number of specific tasks that may be performed by different medical practitioners, including but not limited to doctors, nurses, technicians, treatment planner, etc. By means of non-limiting examples, such tasks may include consultation by a doctor, obtaining medical imaging or test results, treatment planning by a doctor/technician, treatment preparation by nurse(s) and/or technician(s), treatment execution by a doctor/technician, follow-up by a doctor, etc. The different tasks associated with these procedures may be performed at different locations in different facilities. The process of conducting these tasks may be generally considered as medical procedure workflow.

The methods, systems and apparatus of the present invention include apparatus for marking a surgery site (e.g., location of surgical incision) or a site of other medical action with a patient and/or procedure specific medical marking (e.g., bar code, QR codes, RFID/NFC tag, magnetic stripe, etc.), which may be used in conjunction with a medical workflow system for managing medical procedures and/or actions.

In one embodiment, systems and methods of the present invention ensure the validation of the surgical procedure, such as, for example, the patient awaiting the surgical procedure, and the site or location on the patient where the surgery is to be performed. Preferred embodiments of the present invention can be implemented in numerous configurations depending on implementation choices based upon the principles described herein. Various specific aspects are disclosed, which are illustrative embodiments not to be construed as limiting the scope of the disclosure. Although the present specification describes components and functions implemented in the embodiments with reference to standards and protocols known to a person skilled in the art, the present disclosure as well as the embodiments of the present invention are not limited to any specific standard or protocol.

A medical protocol or workflow may also be established which makes the scanning or reading of the medical tag or marking an integral part of the surgical method. Through the use of the protocols in accordance with the embodiments of the present invention, medical staff or personnel (including nurses, doctors, administration staff, etc.) may visually or electronically (e.g. a well know bar code reader or other scanning device) read the identifying markings on the medical tag so as to check and/or confirm the applicable medical action to be taken before the actual medical action (e.g., surgery) is performed. The protocol and system described herein facilitates the performance of certain checks, and establishes a verifiable path to decisions about the patient, the surgical site on the patient, and the surgical procedures. Thus, the medical personnel are guided to correct information associated with the patient and the corresponding surgical procedure.

In an exemplary use of the present invention, when a patient is seen, admitted, etc., a “medical tag” may be associated with the patient's physical chart and the patient himself or herself. All relevant information is associated with the medical tag, including the patient's blood type, relevant medical procedures, lists of known allergies, etc. It does not need to be a full medical record, as any person skilled in the relevant art will understand that some relevant information can be added or removed. Moreover, any relevant information may be associated with the medical tag through the applicable markings. In the case of a specific surgery, the location of where the medical action is to be performed is then associated with the patient's records as well. Thus, the embodiments of the present invention allows medical staff or personnel to enter pertinent information about the patient and the associated procedure to be performed. It may also provide an opportunity to enter vital information for the successful outcome of the surgery including, but not limited to, a patient's medical records, lab tests, x-rays, charts, required prosthetic devices, required surgical kits, staffing requirements, date and time, etc. In a hospital or other healthcare facility where several operating rooms are used simultaneously, embodiments of the present invention may also allow the entry or creation of a listing of the determined time and place of the surgical procedure in addition to entered information, materials, and staff requirement assignments (e.g., needed presence of anesthetist).

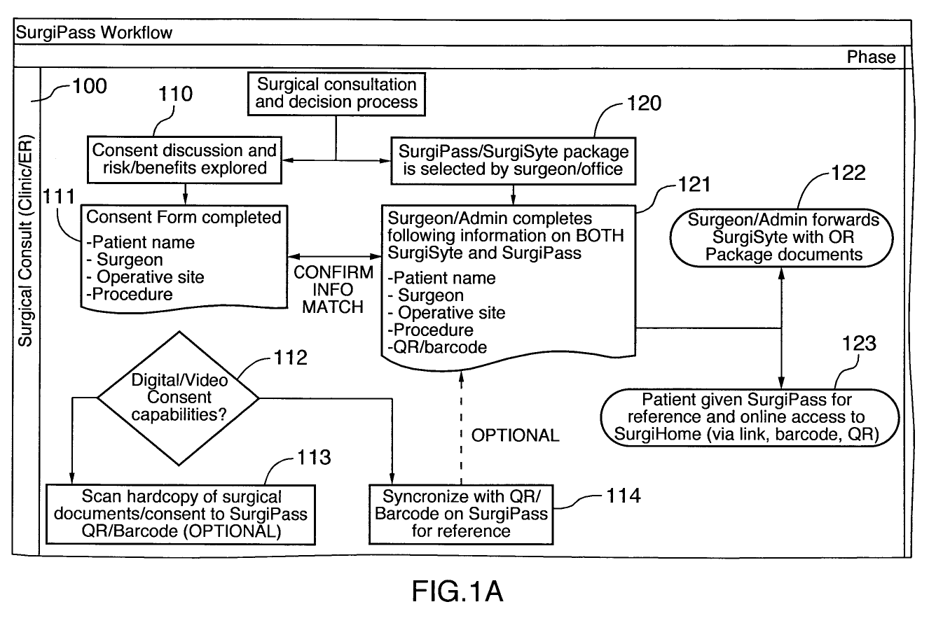

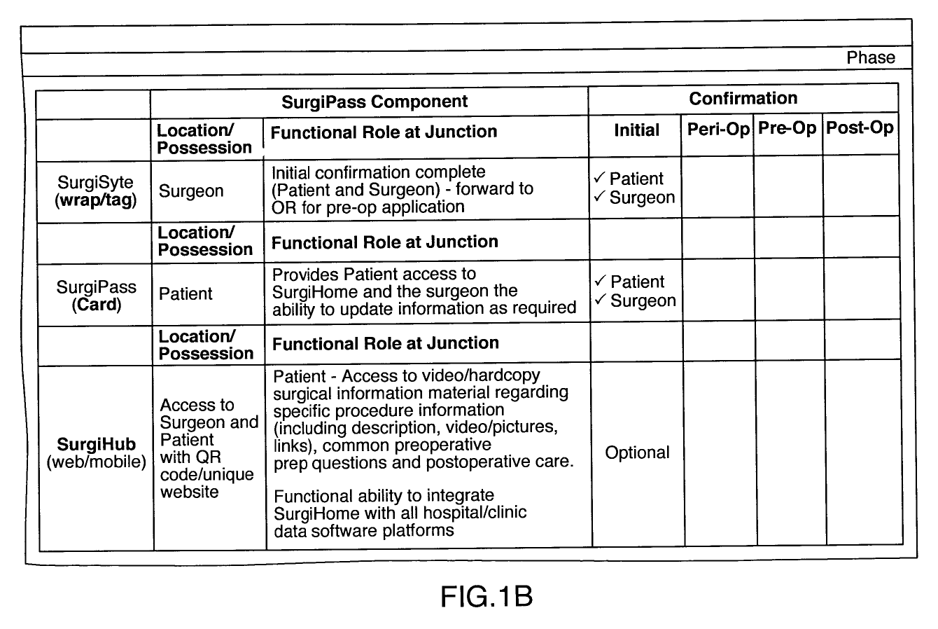

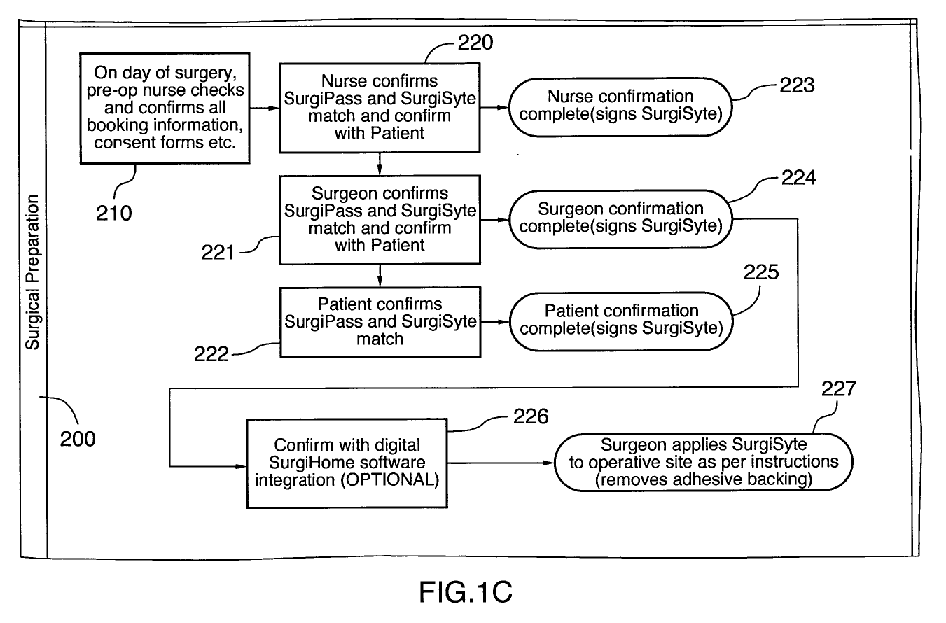

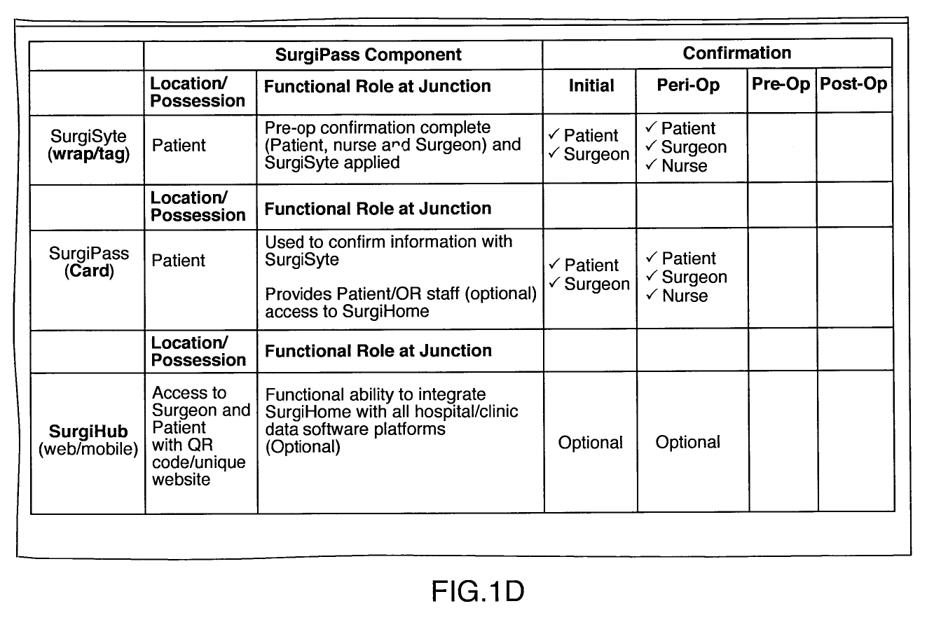

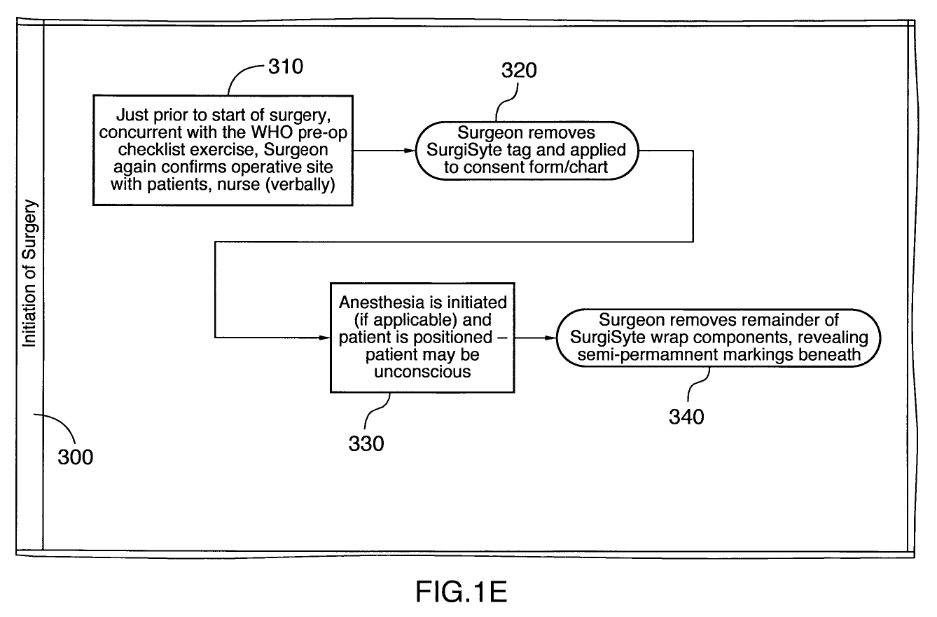

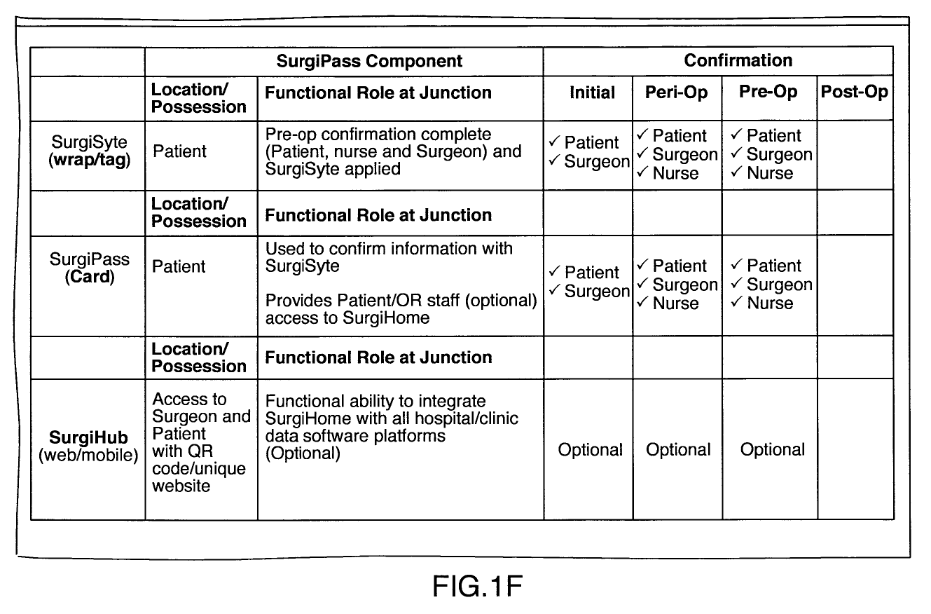

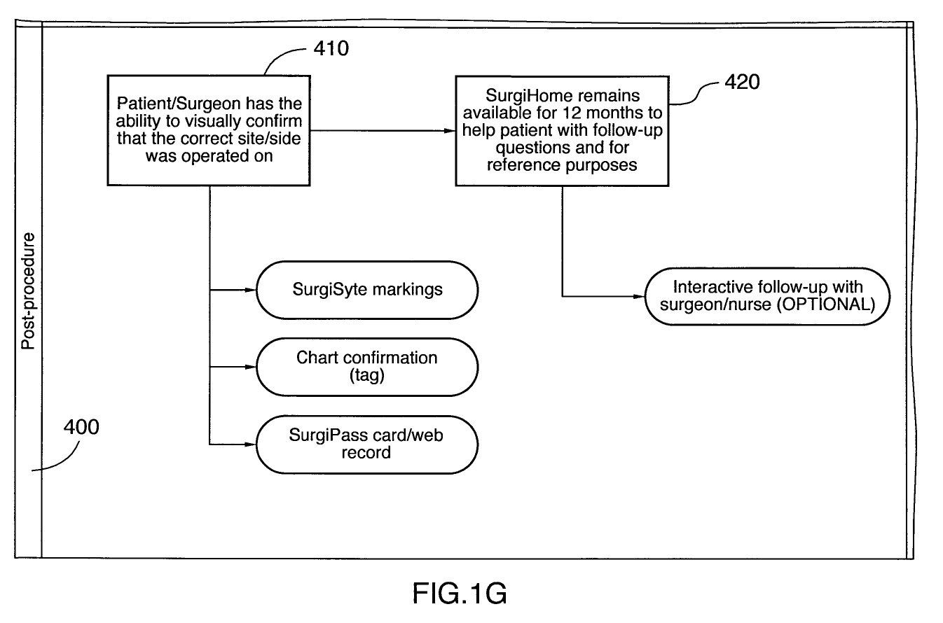

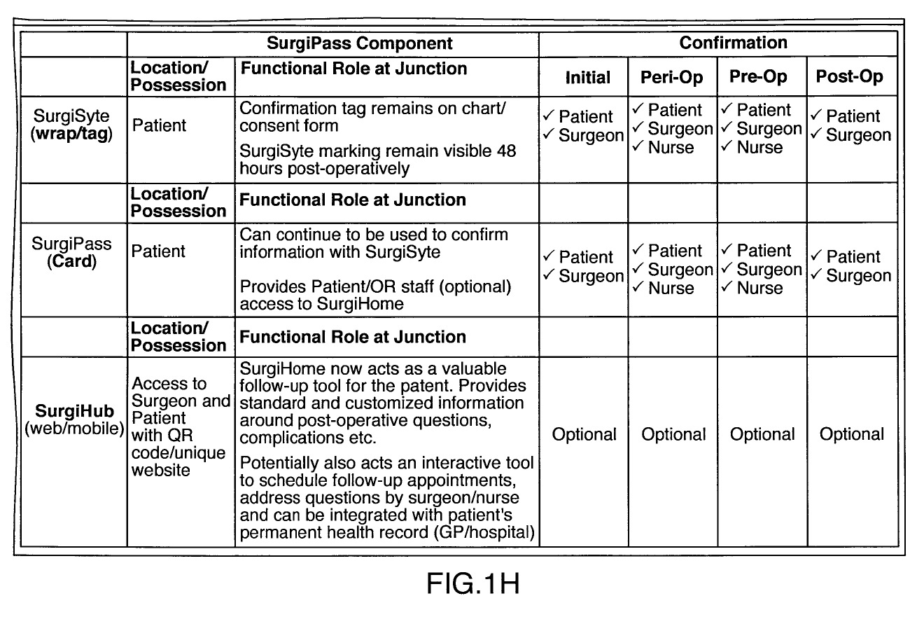

In an embodiment of the present invention, FIG. 1 shows a workflow medical procedure process (e.g. SurgiPass Workflow™) involving consultations, pre-op and post-op procedures, as well as the medical procedure itself. As shown in FIG. 1, there is provided a left portion which outlines the various steps of the workflow embodiments of the present invention and a right portion which sets out the location, role/function and confirmation status of the various SurgiPass components of the present invention (see below). In a preferred embodiment, the medical personnel discuss with patient the consent requirements along with the risks and benefits of the specific procedure that is to be performed (see 110) prior to the planned surgical procedure, commonly referred to as the “consult” 100 (or “surgical consult” in the context of an operation that may be performed). Such a discussion may happen well before or at the time of the procedure. The medical personnel obtain the required personal and medical information of the patient and input the relevant information (including, but not limited to, patient name, type and location of surgery, consent status, appropriate medical personnel (e.g. a surgeon), hospital bar code or other patient identifier, etc.) into the system of the present invention (see steps 111 to 114). It will be understood by a person skilled in the art that the information may be obtained, created, provided and/or recorded in a number of known ways, including, but not limited to, video, audio, hand written or electronic. This can be also synced with other embodiments of the present invention (see 113 and 114).

During the surgical consult 100, the medical practitioner obtains a preferred embodiment of the present invention, such as the SurgiPak™ package (as described in greater detail below) (see step 120). At step 121 of FIG. 1, the required personal and medical information of the patient is transferred to both a patient medical tag identifier and an administration information component of the present invention. In a preferred embodiment, the information provided during the consult may be confirmed to match in both the patient medical tag identifier and the administration information component. The two components of the preferred embodiment of the present invention are then provided to either the patient (SurgiPass™) (see step 122) or the facility administration for the medical procedure (SurgiSyte™) (see step 123).

In a preferred embodiment, the system of the present invention includes a medical tag system. The information obtained during the consult is then transferred to the medical tag system of the present invention. In a preferred embodiment, the medical tag system of the present invention has one or more components; a first component is to be located or directly associated with the patient, while the second component is to be used with and/or where the procedure or activity is to take place (e.g. hospital, clinic, etc.). The patient identification and information component is referred to herein as the patient medical tag or patient medical tag identifier, while the second component is referred to herein as the administration information component. The required personal and medical information of the patient is transferred to both the patient medical tag identifier and the administration information component. In a preferred embodiment, the information provided during the consult may be confirmed to match in both the patient medical tag identifier and the administration information component. In a further preferred embodiment of the present invention, electronic copies of the personal and medical information of the patient may be copied, created or provided digitally and/or electronically. The electronic medical records may be stored, entered and created in any applicable database that would be familiar to those skilled in the relevant art. Embodiments of the medical tag system of the present invention will be discussed in greater detail below.

When the medical procedure is scheduled to take place, a medical practitioner, such as, for example, a pre-operative (“pre-op”) nurse, checks and confirms that all necessary information has been obtained and is contained in the medical record (whether electronic of paper based), including but not limited to, booking information, consent forms, etc. (see 210). Step 210 may be completed on the day of surgery, or close to the surgical procedure/time. Booking information may include information sent by the surgeon including patient name, surgical procedure, surgical site/side, specific equipment requirement etc. Typical example of booking forms for both emergency and elective procedures are known in the art. It will be understood by person skilled in the relevant art that the booking forms, consent forms, etc. have a variety of mandatory and non-mandatory information, including but not limited to, personal information, date, time of booking, applicable codes, surgical information, special requirements, medical history, current medical conditions, etc. (for example, see step 220). The pre-op medical practitioner may confirm that the patient information or data provided on or in the patient medical tag identifier (referred to SurgiSyte™) to be located or directly associated with the patient, is correct (see steps 221 to 225). One or more pre-op medical practitioners may also confirm that the patient information or data provided on or in the administration information component (referred to SurgiPass™ data card) is correct (221, 223). The patient may also confirm that the information is correct (222, 225). SurgiPass™ data card refers to the card given to the patient containing a patient's unique number pertaining to the specific surgical procedure and optional space for additional data labels to be printed containing patient name and admin data, surgical date/site, facility etc. Upon completion of this task each pre-op medical practitioner may, in a preferred embodiment, indicate on one or both of the SurgiSyte™ and/or the SurgiPass™ that the check has been completed (see steps 221 to 225). This may be done by signing (electronic or otherwise) one or both of the first and/or second components of the system or any other manner know to a person skilled in the art. In a preferred embodiment, additional medical practitioners (such as, for example, a surgeon) may also confirm that the information provided in the SurgiSyte™ and/or the SurgiPass™ is correct and that the check has been completed. In a more preferred embodiment of the present invention, the patient may also confirm that the information provided in the SurgiSyte™ and/or the SurgiPass™ is correct and that the check has been completed. Upon completion of this task the additional medical practitioner(s) and/or the patient may, in a preferred embodiment, indicate on one or both of the SurgiSyte™ and/or the SurgiPass™ that the check has been completed. This may be done by signing (electronic or otherwise) one or both of the first and/or second components of the system or any other manner know to a person skilled in the relevant art. Once the above noted steps are completed, a medical practitioner (in a preferred embodiment a surgeon) would apply the SurgiSyte™ to the applicable location of the activity (see step 227). It will be understood that the SurgiSyte™ may be affixed to the patient in any manner know to a person skilled in the relevant art including but not limited to, a pressure sensitive adhesive of the type suitable for adhesion to human skin and releasable from the skin without injury. In an emergency situation 100, 200 and 300 can occur very close to each other (in the order of hours) whereas in an elective situation the timing may vary significantly.

In a preferred embodiment, the SurgiSyte™ may include a surgical decal may be created by preprinting a clear plastic substrate with an ink specially formulated to adhere to the plastic and then covering the printing with a backing. The ink may be removed easily and transferred to the patient's skin once applied. On the skin, the ink remains somewhat permanent; it will not wash off, smear or fade in less than, for example, ten days. Thus, the “tattooed” image on the patient's skin remains sharp and consistent and visible on all skin tones The ink color used may be dark (e.g., black) and could even be readable by the bar code scanner, even where contrast would be difficult on patients with skin of very dark color. It will be understood that various medical inks can be used. Such ink would be accepted or approved for use in surgical or medical procedures (e.g. hypoallergenic, biodegradable).

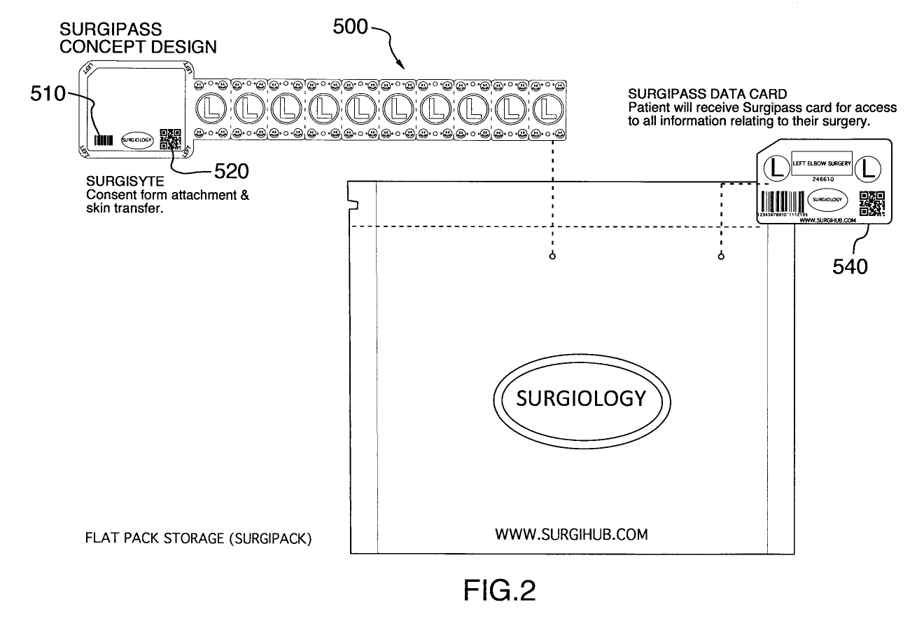

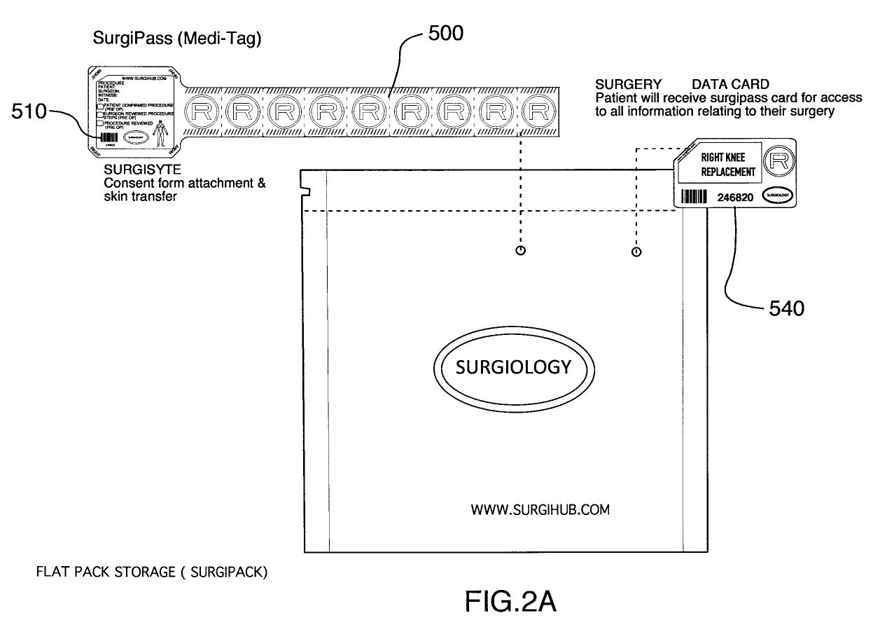

In order to simplify the system for medical practitioners, the various components of the present invention can be provided in a packaged set (see FIG. 2). In a preferred embodiment, the SurgiPack™ package can comprise both the SurgiSyte™ and/or the SurgiPass™ as well as other components of the present invention, such as, for example, software that is employed in the present invention.

In a preferred embodiment, there is provided a medical tag that has multiple elements, including a decal. These elements are provided in both machine-readable medical marking (e.g., barcode, QR codes, RFID/NFC tag, magnetic stripe, etc.) and human-readable form. The first copy of the number (first part of decal) is a barcode which may be applied to a patient's physical medical chart. The second copy (second part of decal) of the number may be removed and maintained by an operating room nurse or a person in charge of operating room administration for the purpose setting-up the operating room facilities and resources prior to surgery. In the pre-operative visit, the surgeon may take the third part of surgical decal (third copy of number) and apply it directly to the patient at the site at which the surgery is to be performed (i.e., on the skin of the surgical site), potentially utilizing cross hairs to mark the site on the patient where the surgical procedure is to be performed. In one embodiment, this section of the decal is applied to a patient in any manner know in the relevant art. In a preferred embodiment, this can be accomplished by removal of the protective adhesive layer, by wetting and pressing it onto the patient's skin, or any other process known in the art. The surgical decal may include a place for the surgeon to mark and/or sign (e.g., with a felt tip pen).

At the commencement of the medical procedure 300, and preferably concurrent with the current protocol used by the hospital, such as for example, that provided in the WHO pre-op checklist provided currently at http://www.who.int/patientsafety/safesurgery/tools resources/SSSL_Checklist_finalJun08.p df, the medical practitioners can confirm the operative site based on the location of the SurgiSyte™. (see step 310). Once the location of the medical procedure (e.g. surgery) is confirmed, a medical practitioner may remove the SurgiSyte™ which has been attached to the patient at the location indicated (see step 320). As described, a portion of the SurgiSyte will function as a permanent record for the patient's chart, for example, the removed SurgiSyte™ now can be applied to the consent form (e.g. using the pressure sensitive adhesive) or chart (paper) or may be recorded in an electronics matter. In a preferred embodiment, there may be a semi-permanent marking or medical ink transferred to the skin of the patient near the location of the procedure (see step 340).

Once the medical procedure in complete (e.g. the post-op stage 400), the patient and the medical practitioners have the ability to visually confirm that the correct site/side has been operated on (see steps 410 and 420).

In another aspect of the invention, a web based application or other suitably secure computer database can be provided as a means for storing the information and providing follow-up to the patient. In a preferred embodiment, the information from the medical tag system of the present invention and the various steps of the workflow described in the present application can be made available remotely or otherwise away from the medical location. An advantage of the system would be that information could be changed up to and including during the operation so that the latest and more up-to-date information is always available to those who depend on it. The patient's HR/EHR may be stored in a local or remote database, or in any other local or remote storage medium. The accessed record may then be displayed via a monitor, smart phone, tablet or other remote device.

In yet another embodiment, the medical marking may be applied using techniques other than decals. For example, the medical marking may be applied to the patient using a computer-controlled printing apparatus such as an ink-jet printer, a plotter, a tattooing machine or the like. In addition, in one embodiment of the present invention, the topology of the site of the medical action is determined such that a modified bar code can be created that causes the printed bar code to appear linear despite being applied to a non-linear or planar surface.

In a preferred embodiment of the present invention, the medical tag (in a preferred embodiment SurgiSyte™ 500) and its applicable markings are set out in FIG. 2. The marking set out in FIG. 2 may comprises a pattern of graphics in a particular lay out design to properly alter the medical practitioners. In the preferred embodiment as shown in FIG. 2, there may be provided markings 510 (e.g. bar codes or QR codes, RFID/NFC tag, magnetic stripe, etc.), along with colour and/or location codes for applicable locations 520 on or in the body.

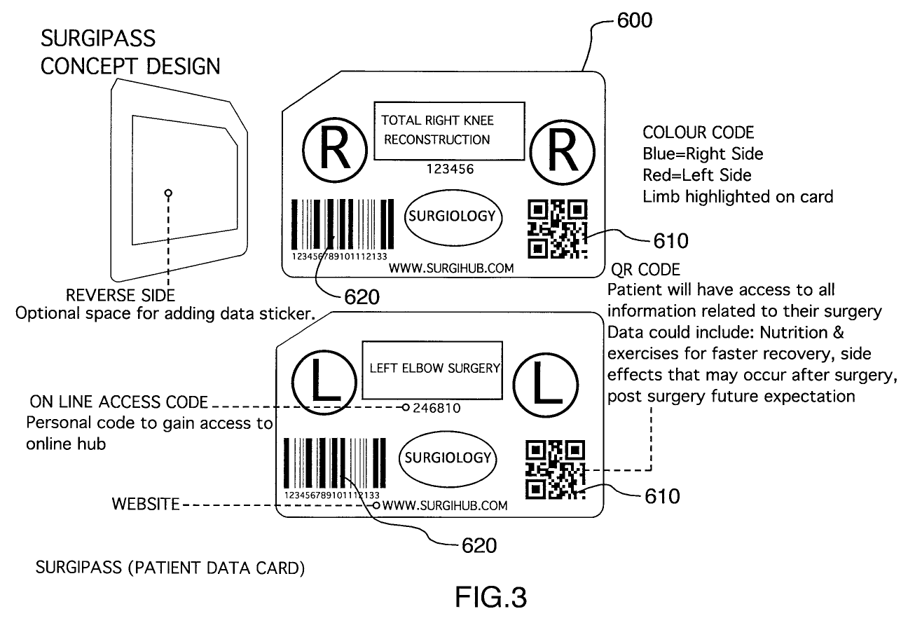

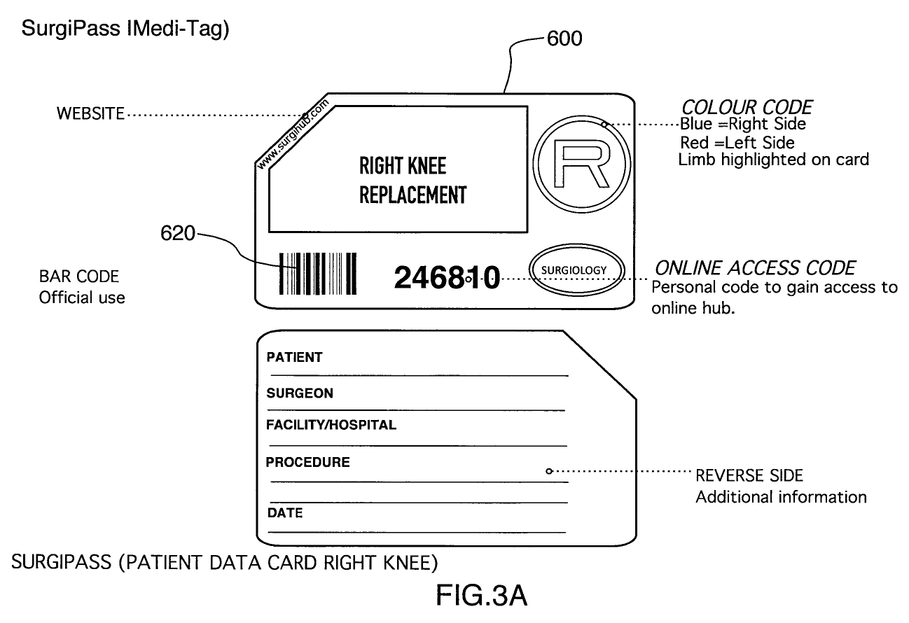

It will be understood by a person skilled in the relevant art that, for small and hard to see or reach areas, SurgiSyte™ itself, the decal and the machine-readable medical markings (510, 520) can be printed at various sizes. For example, the same imagery may be printed at half-size for images required for hands or fingers. As illustrated in FIG. 2, there is provided an embodiment of the present invention in which the patient identification and information component 500 is provided. There is a first portion 520 in which is provided the surgical site detail as well as markings specific to the patient, surgical procedure, surgical site, medical facility location, etc. As shown in FIG. 2, 500 may be colour coded to correspond to a particular side and/or location (see legend 220 in FIG. 2). As shown in FIG. 2, there is also provided a preferred embodiment of the administration information component 540 (e.g. SurgiPass™). FIG. 3 and the description below provides a more detailed description of SurgiPass™.

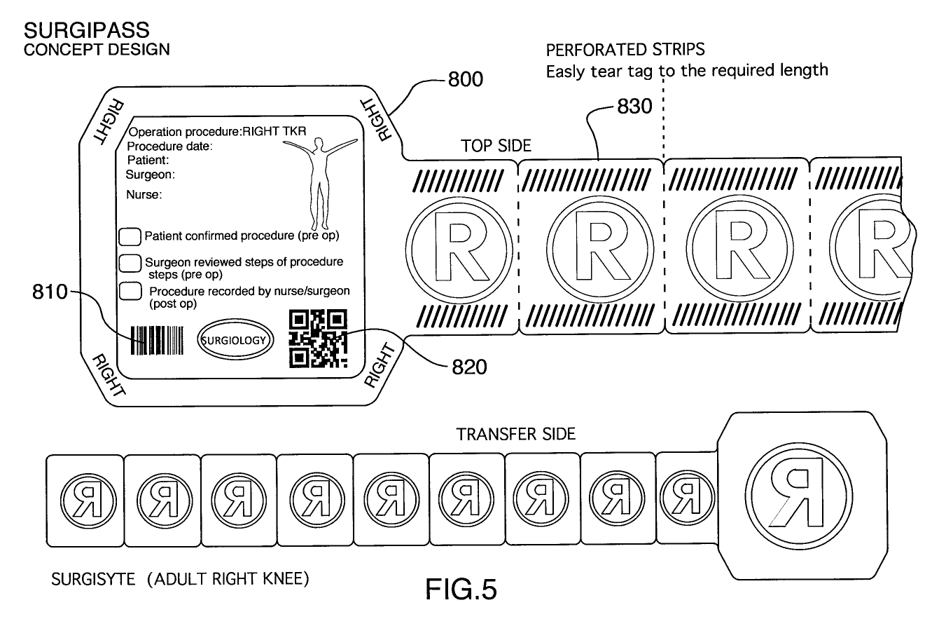

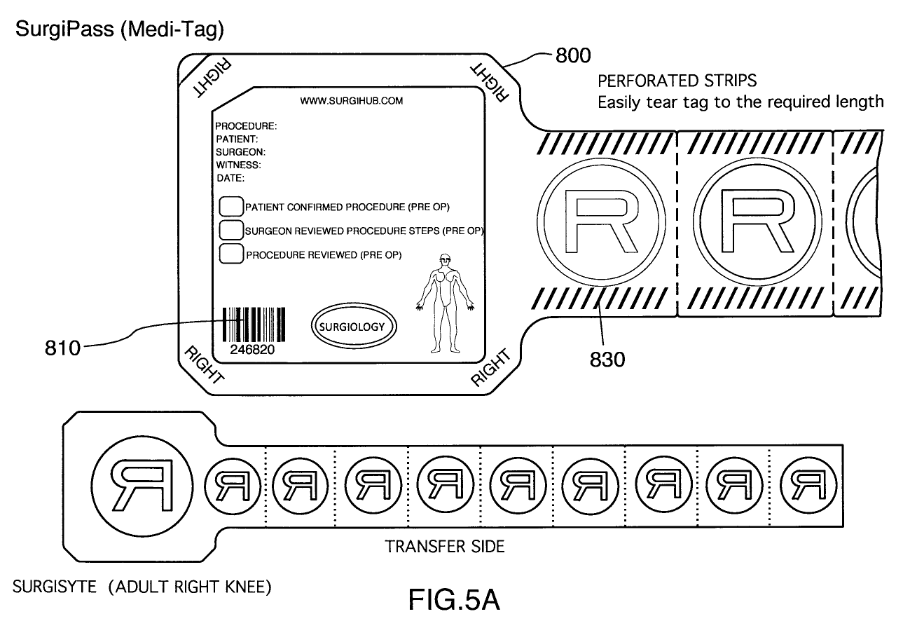

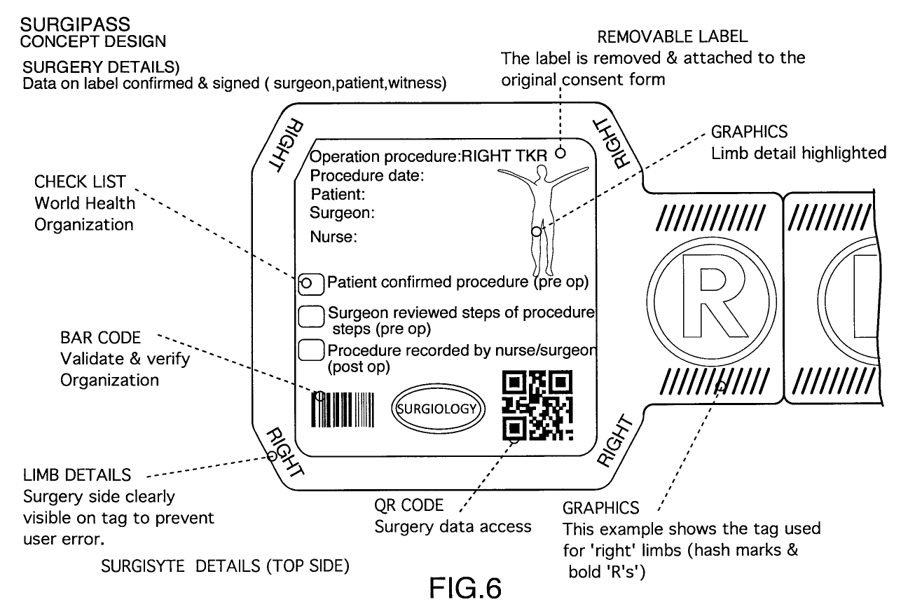

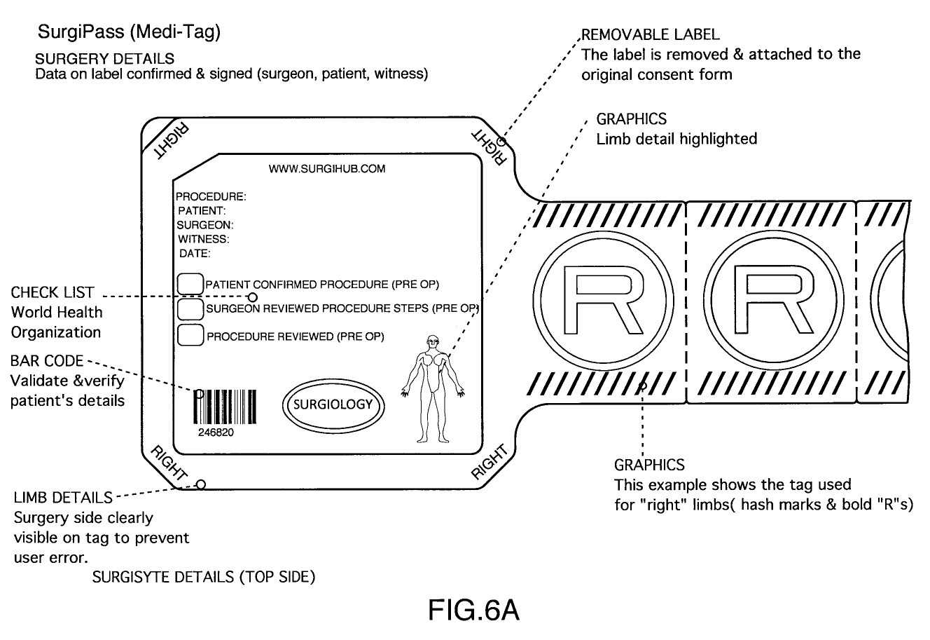

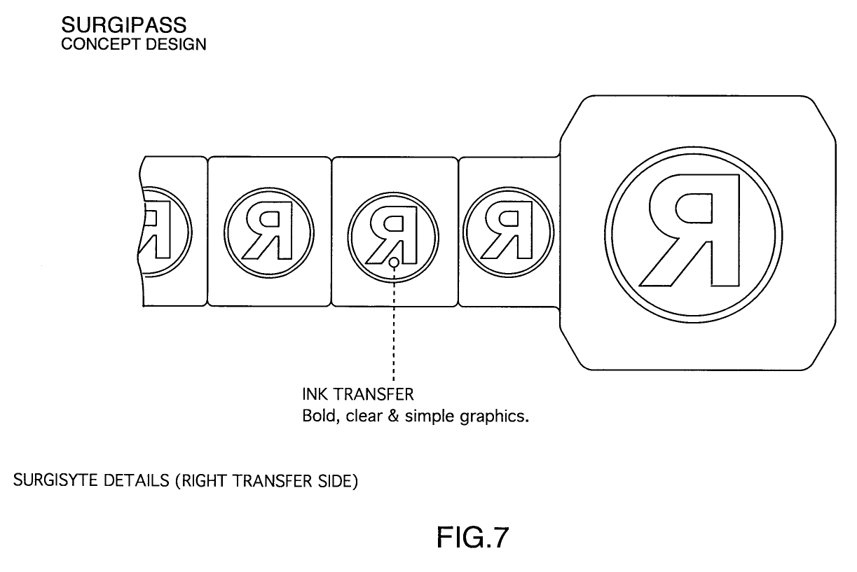

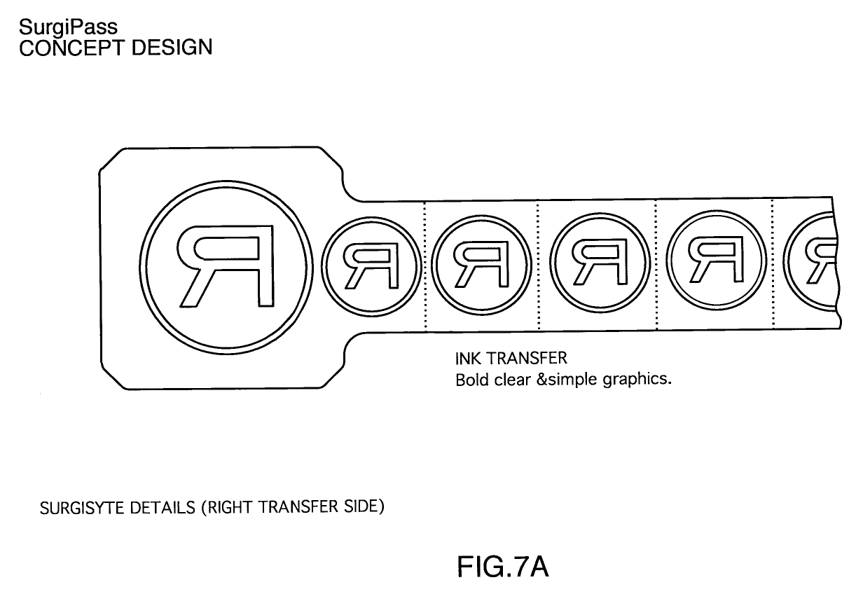

The SurgiSyte™, in a preferred embodiment, has a strap portion that is sized to be wrapped around the site of the surgical procedure (530). In FIG. 2, there is provided a strap that can be wrapped around the left arm, typically around the wrist. As it is designed to be wrapped around the left arm, it is red, which in this embodiment indicates the left side. It will be understood by a person skilled in the relevant art that the specific colour is not relevant as any choice of color can be used provided that the color are consistent with legend 220. FIGS. 5 and 6 provide a further embodiment where a SurgiSyte™ for the right side is provided 800. In FIG. 7, the underside (or transfer side) of the SurgiPass™ is shown. The underside provides the material that will be transferred to the skin of the patient.

In FIG. 3, there is provided a preferred embodiment of the SurgiPass™ 600. As can be seen in FIG. 3, there is provided the same color coding as provided in FIG. 2. There is also provided markings and/or embedded machine-readable encoding (e.g. barcodes and QR codes, RFID/NFC tag, magnetic stripe, etc. 610 and 620) that correspond to the relevant information of the patient, procedure, facilities, etc. The barcode or QR/NFC/RFID etc. information will include or provide digital access to all basic patient information, surgical procedure site and facility location, surgeon etc. This will also allow a direct link to the patient's SurgiHub site with further detailed information regarding pre/post-op care, surgical procedure explanations/video, risks/benefits and a direct link to the medical team to clarify any additional questions and provide direct or remote follow-up care as applicable. The SurgiHub™ site may also provide peer-to-peer support for the patient and/or the medical professionals involved in the care pathway.

Elements of the present invention may be implemented with computer systems which are well known in the art. Generally speaking, computers include a central processor, system memory, and a system bus that couples various system components including the system memory to the central processor. A system bus may be any of several types of bus structures including a memory bus or memory controller, a peripheral bus, and a local bus using any of a variety of bus architectures. The structure of a system memory may be well known to those skilled in the art and may include a basic input/output system (BIOS) stored in a read only memory (ROM) and one or more program modules such as operating systems, application programs and program data stored in random access memory (RAM). Computers may also include a variety of interface units and drives for reading and writing data. A user or member can interact with computer with a variety of input devices, all of which are known to a person skilled in the relevant art.

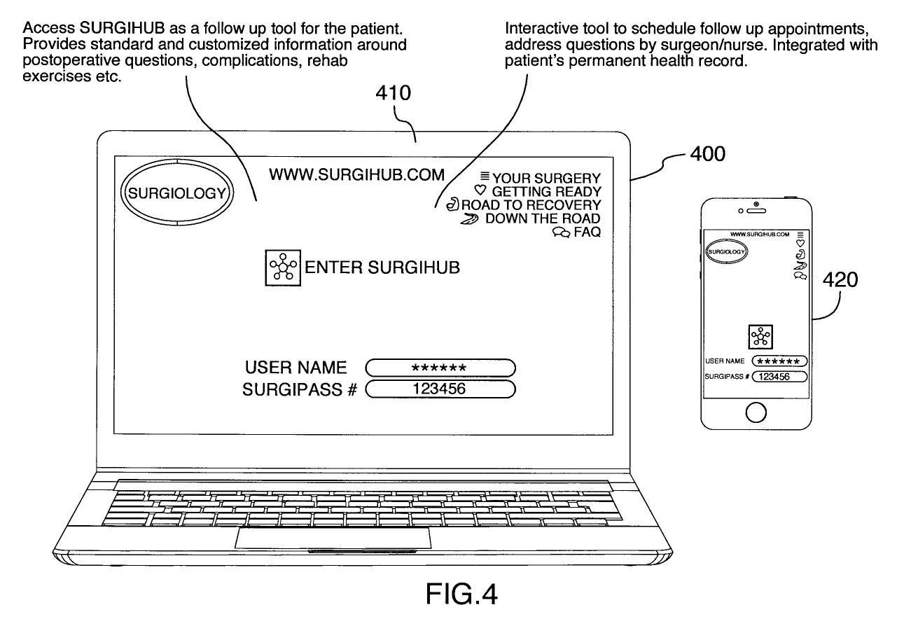

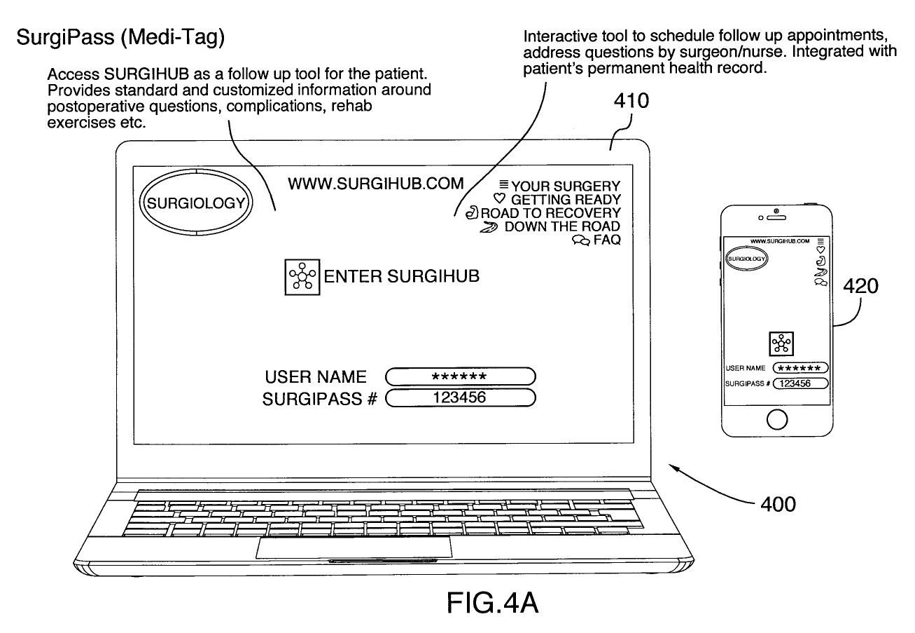

In FIG. 4, there is provided a preferred embodiment of the computer system 470 (e.g. laptop 450 or mobile device 460) that may be used with the medical tag system of the present invention (SurgiHub™).

In one embodiment of the invention, a scanning device (not shown) used for reading the information provided in the system of the present invention and described above may be connected to a computer in the operating room or another location. The connection may be either wired or wireless, depending on the configuration of the room in which the device is to be used. Moreover, the device may be a wand-style scanner, a gun-style scanner, a flat-bed scanner or any other style scanner that reads bar codes or other machine-readable technology.

The active monitoring of the medical action need not be performed by a laptop computer 400 (See FIG. 4, for example). Instead, a separate device, such as a handheld device (optionally with a display), such as a mobile device (e.g. smart phone 410) and an internal processor may act as an integrated scanner and controller. The information about the patients of the day and the medical actions and/or procedures to be performed thereon can be downloaded to the handheld device. Furthermore, while the above description has been provided in terms of a bar code scanner reading a medical marking, the teachings of the present invention may additionally be used in conjunction with other machine-readable medical tags carrying information. One example of such a machine-readable medical tag is an RFID tag such as may be embedded into the SurgiSyte™. Moreover, a combination of reading devices can be used such that the patient is identified by a machine-readable medical tag while the surgical equipment and test results are encoded with bar codes such that the reading devices communicate (either with each other or with a central controller) to ensure that the information read from each is consistent.

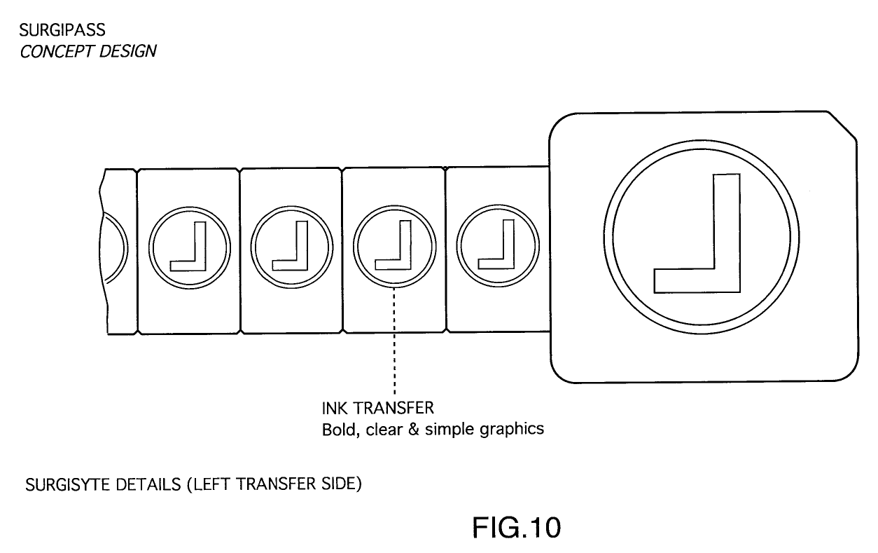

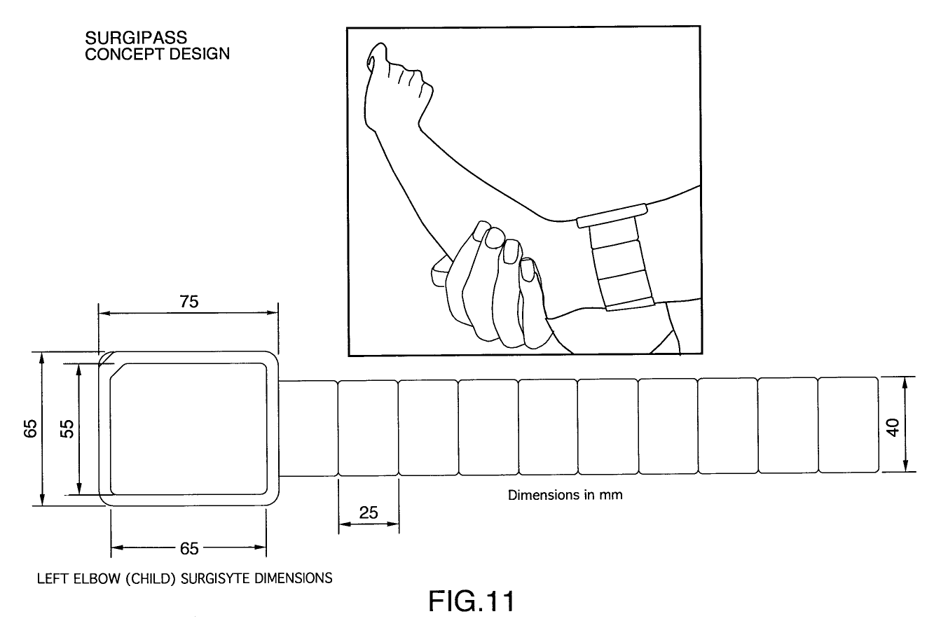

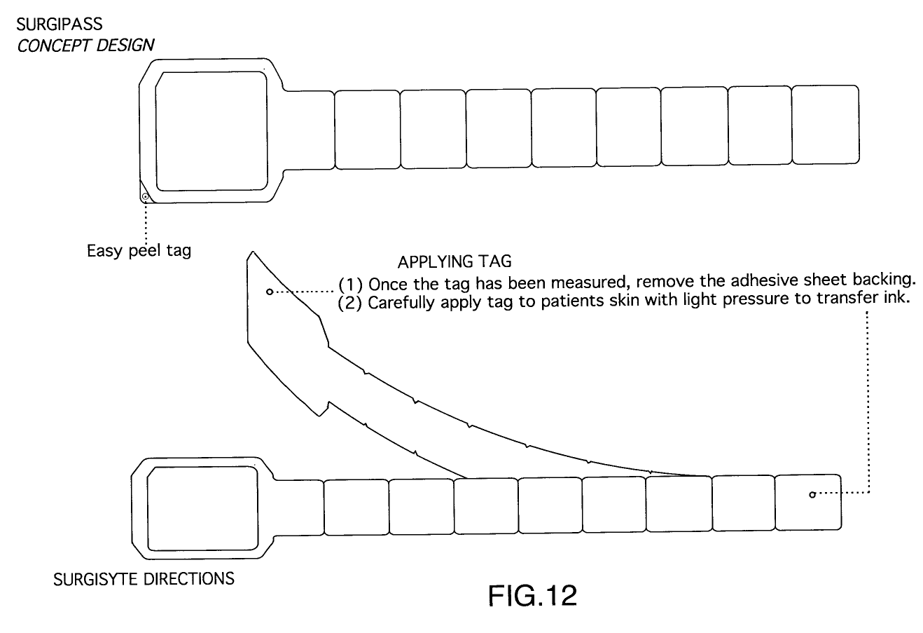

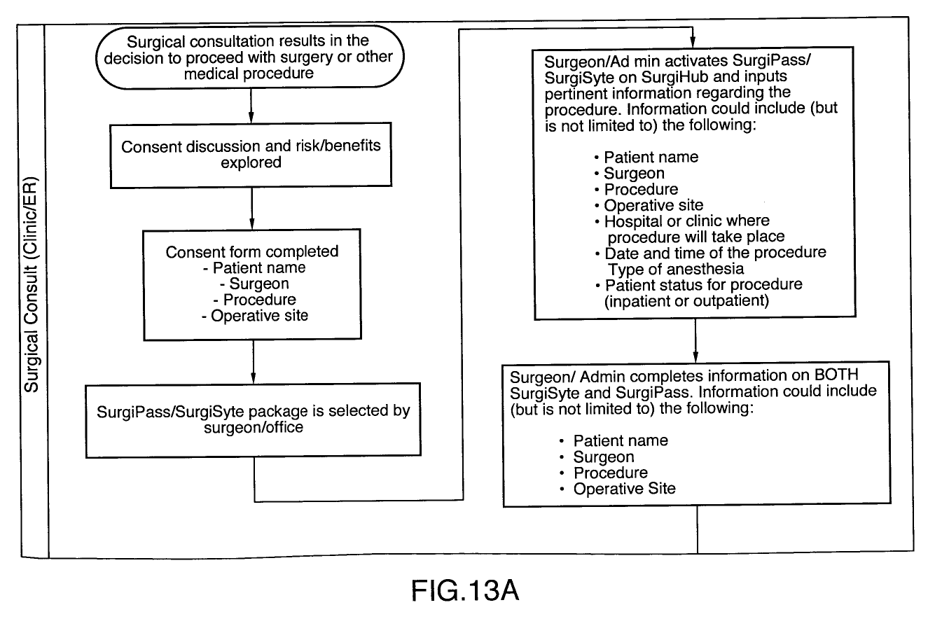

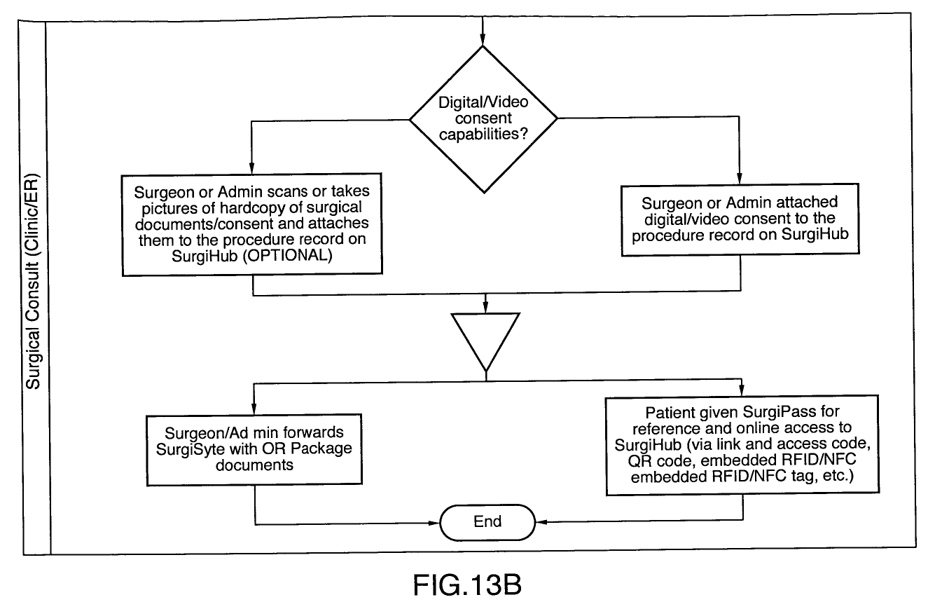

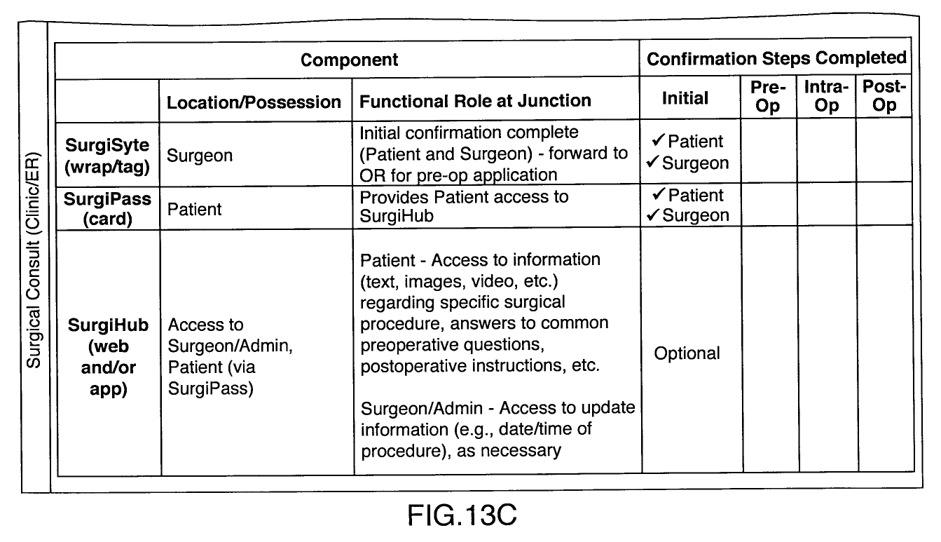

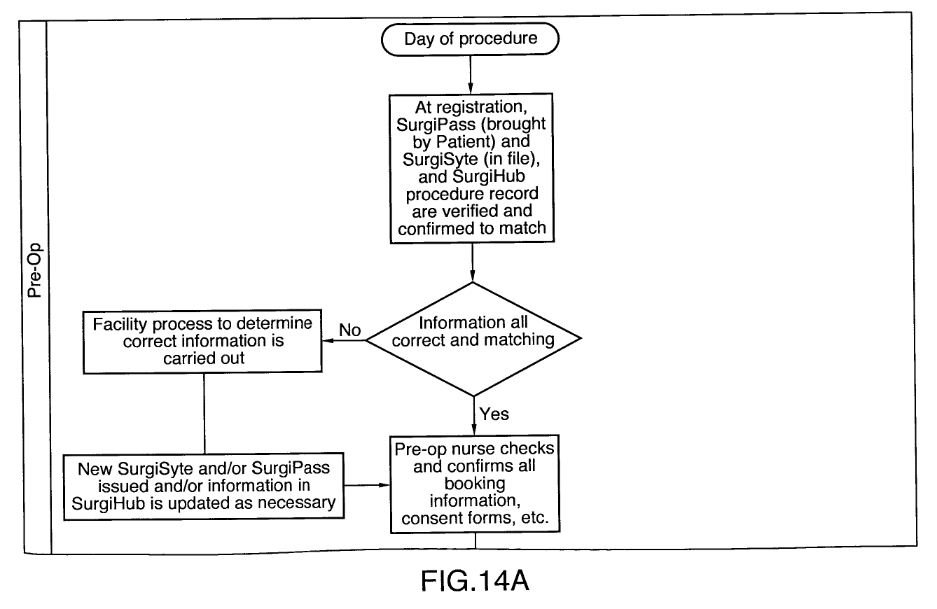

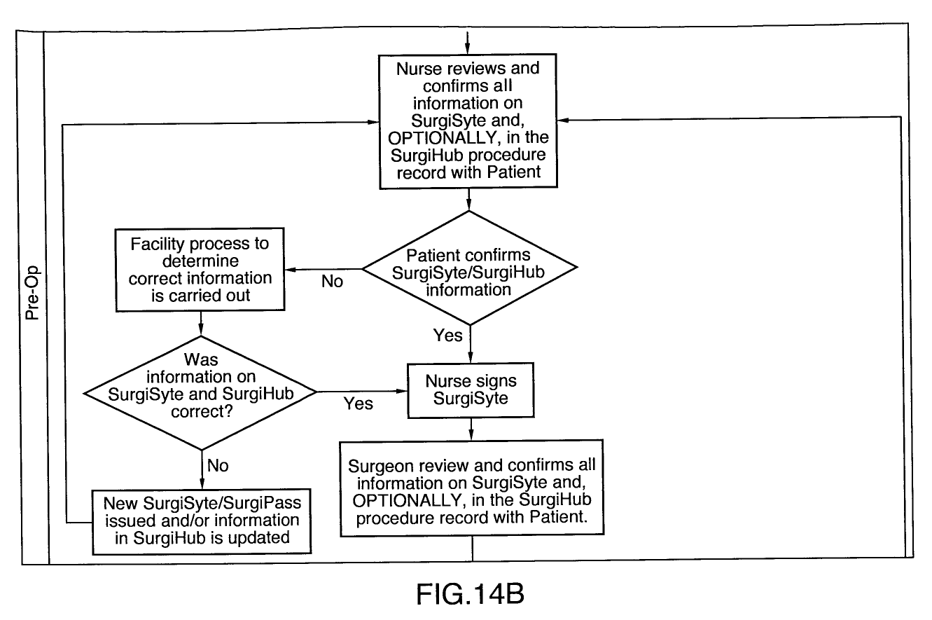

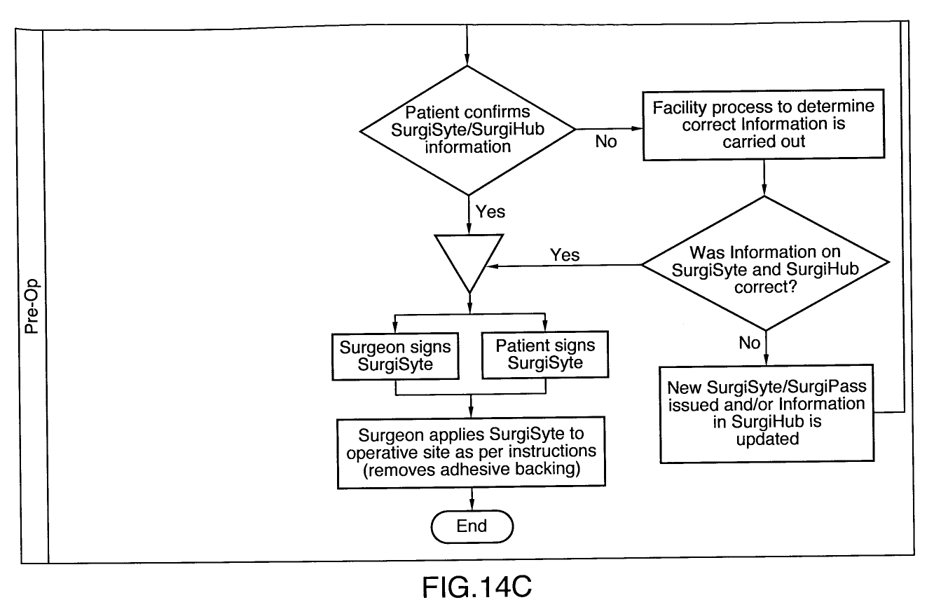

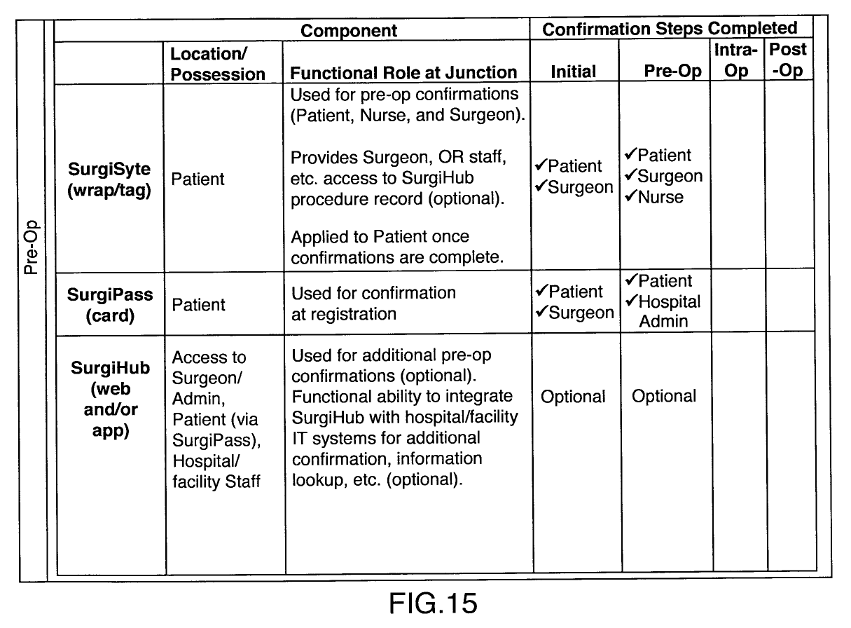

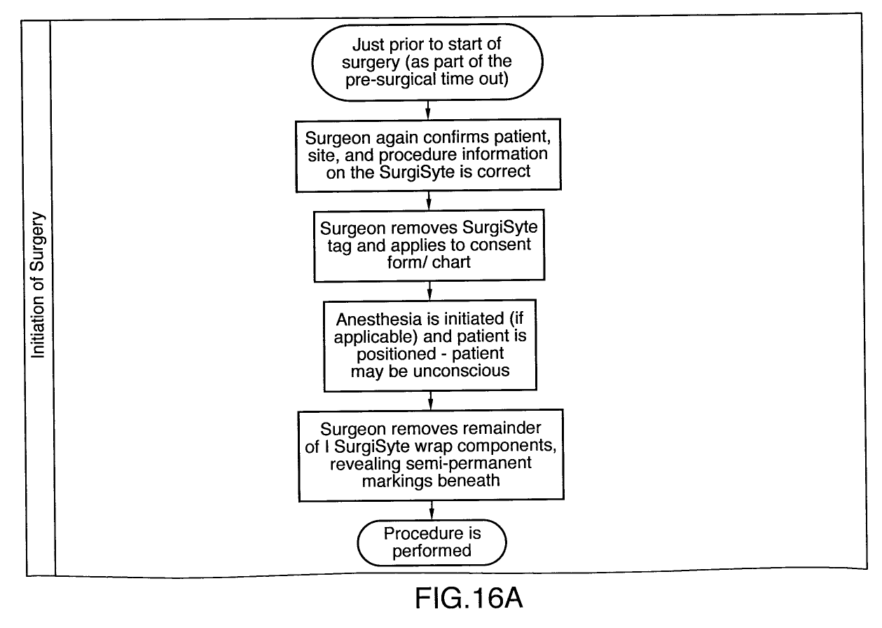

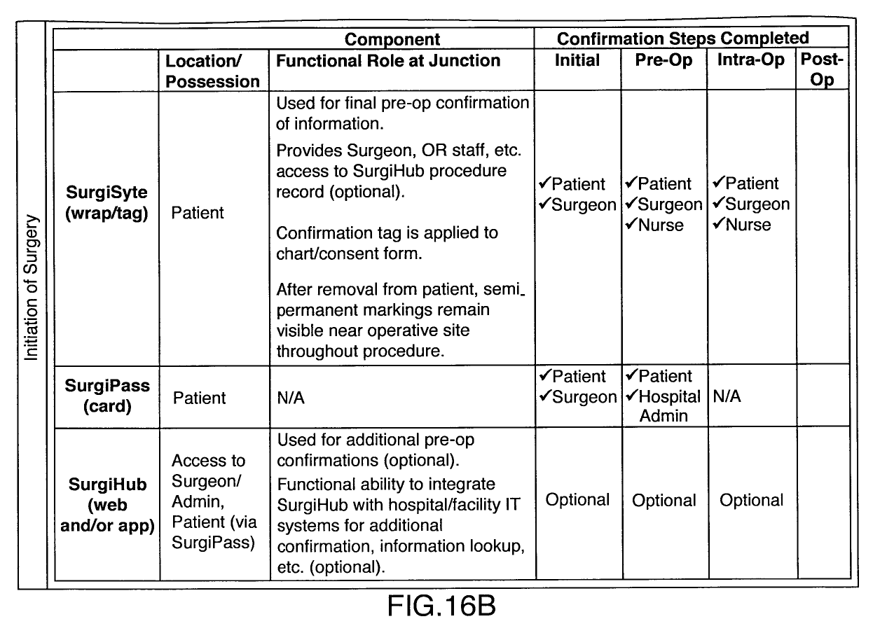

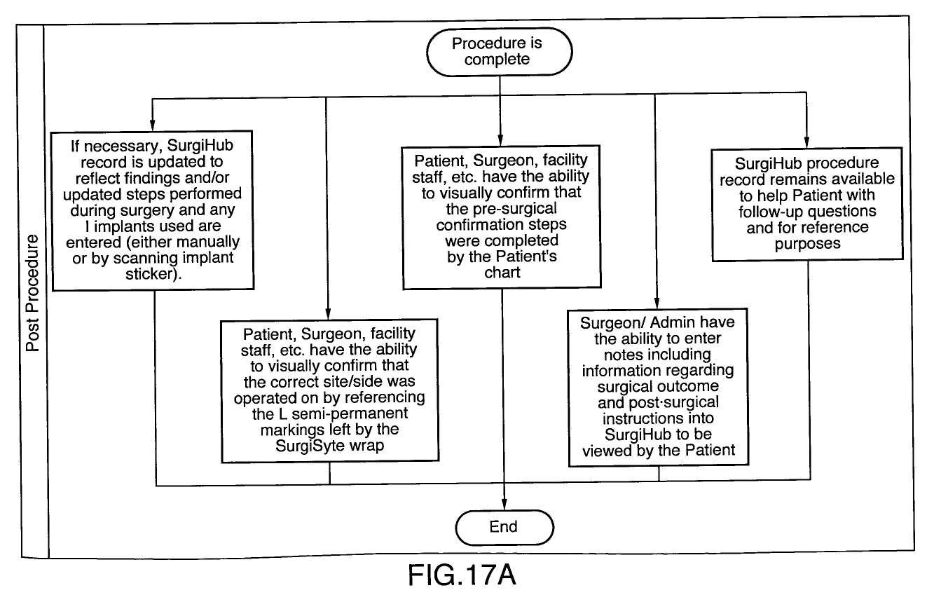

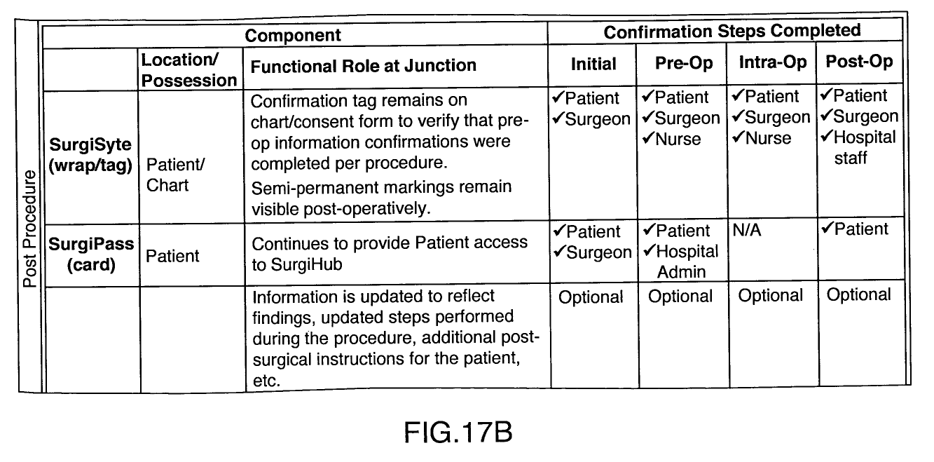

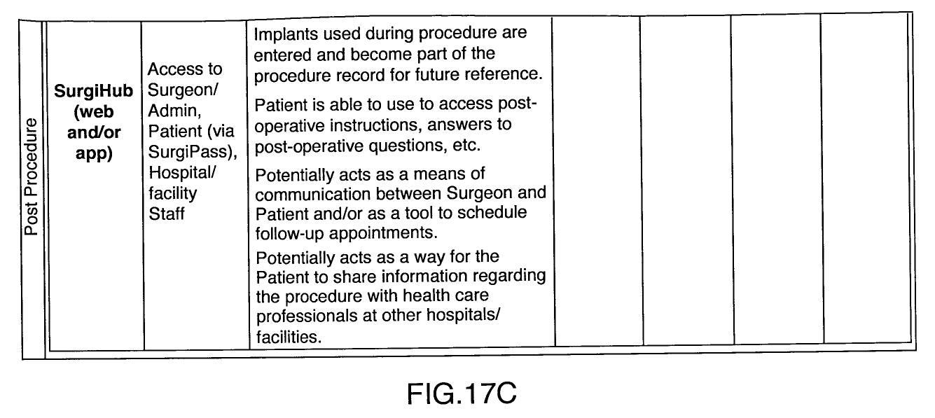

FIGS. 5 to 12 provides further embodiments of the present invention and their operation with the workflow of the present invention. FIGS. 5, 6 and 7 show how the SurgiSyte™ may be used to link with the SurgiPass™ system. In FIG. 5, there is provided a preferred embodiment of the SurgiSyte™ 800. As can be seen in FIG. 5, there is provided the same color coding as provided in FIG. 3. There is also provided markings (e.g. barcodes and QR codes, RFID/NFC tag, magnetic stripe, etc. 810 and 820) that correspond to the relevant information of the patient, procedure, facilities, etc. The barcode information will include all basic patient information, surgical procedure site and facility location, surgeon etc. This will also allow a direct link to the patient's SurgiHub site with further detailed information regarding pre/post-op care, surgical procedure explanations/video, risks/benefits and a direct link to the medical team to clarify any additional questions.

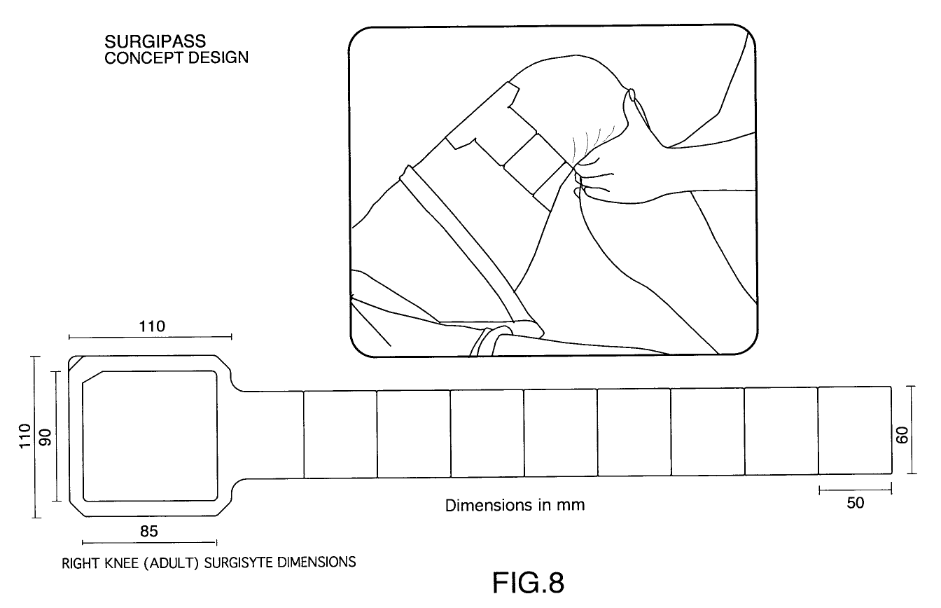

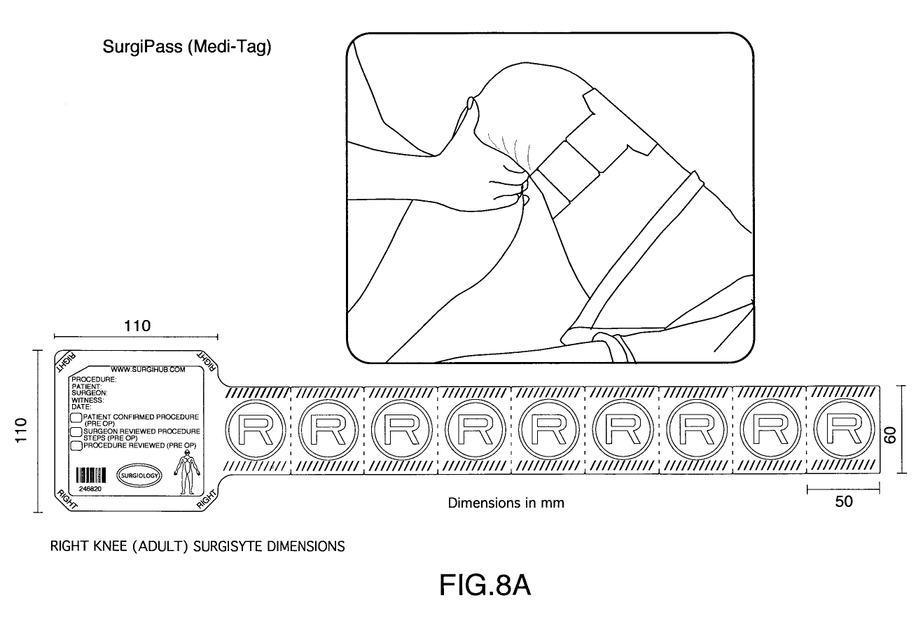

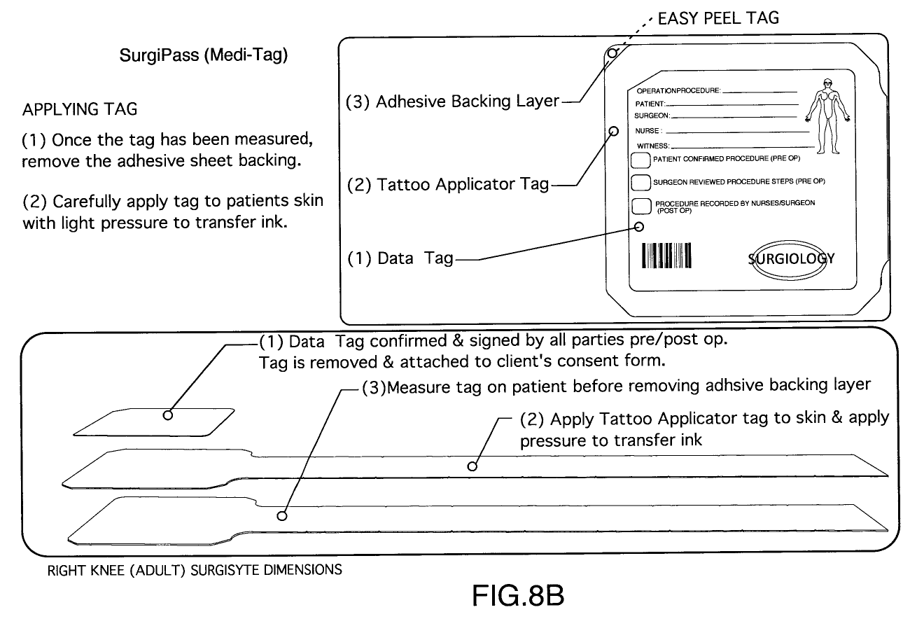

FIG. 8 shows embodiments of the present invention being used in association with the right knee.

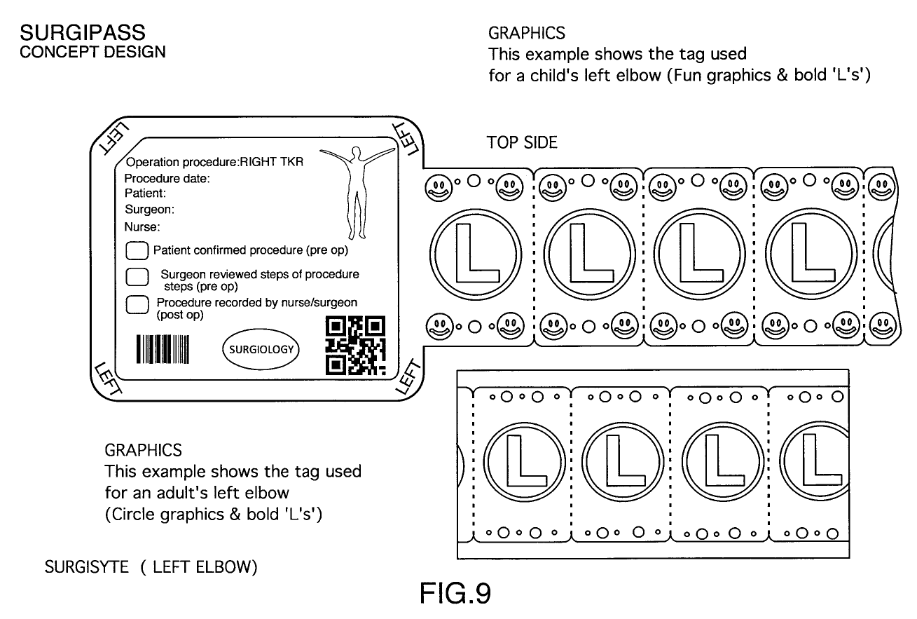

FIGS. 9 to 12 show embodiments of the present invention being used in association with the left elbow of a an adult or a child.

It will be understood by a person skilled in the relevant art that the term “scanner” or “scanning device” refers to any electronic device that can be used to scan a bar code or other electronic or digital marking or any other machine-reading technology. Such a scanner may also be able to access a computer network such as, for example, the internet. Typically, a scanner comprises a display screen, at least one input/output device, a processor, memory, a power module and a tactile man-machine interface as well as other components that are common to scanners individuals or members carry with them on a daily basis. Examples of scanners suitable for use with the present invention include, but are not limited to, bar code readers, smart phones, cell phones, wireless data/email devices, tablets, and PDAs.

One skilled in the relevant art would appreciate that the device connections mentioned herein are for illustration purposes only and that any number of possible configurations and selection of peripheral devices could be coupled to the computer system.

Computers can operate in a networked environment using logical connections to one or more remote computers or other devices, such as a server, a router, a network personal computer, a peer device or other common network node, a wireless telephone or wireless personal digital assistant. The computer of the present invention may include a network interface that couples the system bus to a local area network (LAN). Networking environments are commonplace in offices, enterprise-wide computer networks and home computer systems. A wide area network (WAN), such as the Internet, can also be accessed by the computer or mobile device.

It will be appreciated that the type of connections contemplated herein are exemplary and other ways of establishing a communications link between computers. Mobile devices and networks can be used. The existence of any of various well-known protocols, such as TCP/IP, Frame Relay, Ethernet, FTP, HTTP and the like, is presumed, and computer can be operated in a client-server configuration to permit a user to retrieve and send data to and from a web-based server. Furthermore, any of various conventional web browsers can be used to display and manipulate data in association with a web based application.

The operation of the software embodiments of the present invention may be controlled by a variety of different program modules. Examples of program modules are routines, programs, objects, components, data structures, etc. that perform particular tasks or implement particular abstract data types. It will be understood that the present invention may also be practiced with other computer system configurations, including multiprocessor systems, microprocessor-based or programmable consumer electronics, network PCS, minicomputers, mainframe computers, and the like. Furthermore, the invention may also be practiced in distributed computing environments where tasks are performed by remote processing devices that are linked through a communications network. In a distributed computing environment, program modules may be located in both local and remote memory storage devices.

Embodiments of the present invention can be implemented by a software program for processing data through a computer system. It will be understood by a person skilled in the relevant art that the computer system can be a personal computer, mobile device, notebook computer, server computer, mainframe, networked computer (e.g., router), workstation, and the like. In one embodiment, the computer system includes a processor coupled to a bus and memory storage coupled to the bus. The memory storage can be volatile or non-volatile (i.e. transitory or non-transitory) and can include removable storage media. The computer can also include a display, provision for data input and output, etc. as will be understood by a person skilled in the relevant art.

Although this disclosure has described and illustrated certain preferred embodiments. As shown in FIG. 1, in a second situation, of the invention, it may be to be understood that the invention may be not restricted to those particular embodiments. Rather, the invention includes all embodiments which are functional or mechanical equivalence of the specific embodiments and features that have been described and illustrated.

The embodiments for which an exclusive privilege or property are claimed are as follows:

1. A patient medical information system for use in a medical workflow for conducting a medical procedure on a patient at a location on the patient's body, the patient medical information system comprising:
(a) a first medical information component physically associated with the location of the patient's body and providing readable personal health information of the patient;
(b) a second medical information component associated with the patient and providing readable information; and
(b) a third medical information component associated with a non-patient location and providing readable personal health information of the patient and medical workflow related information.

2. The system of claim 1 wherein the first medical information component further comprises a strap for wrapping around an extremity of the patient.

3. The system of claim 2 wherein the first medical information component further comprises readable markings that readable personal health information of the patient.

4. The system of claim 3 wherein the first medical information component further comprises a patient surgical decal and a patient informational decal.

5. The system of claim 4 wherein the surgical decal is applied to the location of the portion of the body of the patient.

6. The system of claim 5 wherein the patient information decal is applied to the second medical information component.

7. The system of claim 6 wherein the markings are selected from the group consisting barcodes, QR codes, RFID/NFC tags, and magnetic stripes.

8. The system of claim 7 wherein the second medical information component is a card providing the patient access to the third medical information component.

9. The system of claim 8 wherein the third medical information component.

10. The system of claim 9 wherein the third medical information component is a web based application.

11. A medical workflow for conducting a medical procedure on a patient, the medical workflow comprising:
(a) a medical professional and the patient confirming that the medical information of the patient associated with a first medical record component, the first medical record component physically associated with the patient, is correct and accurate;
(b) confirming the site of the medical procedure based on the location of the first medical record component on the body of the patient;
(c) marking the site of the medical procedure based on the location of the first medical record component; and
(d) removing the patient medical tag physically from the site of the medical procedure and transferring the information contained therein to a second medical record component.
